(19) United States
(12) Patent Application Publication
(10) Pub. No.: US 2015/0223838 A1
(43) Pub. Date: Aug. 13, 2015

(54) Articulating Method Including A Pre-Bent Tube

(71) Applicant: Covidien LP, Mansfield, MA (US)

(72) Inventors: Jaroslaw T. Malkowski, Trumbull, CT (US); Eric Taylor, East Hampton, CT (US)

(21) Appl. No.: 14/696,817

(22) Filed: Apr. 27, 2015

Related U.S. Application Data
(62) Division of application No. 13/735,075, filed on Jan. 7, 2013.
(60) Provisional application No. 61/584,713, filed on Jan. 9, 2012.

(51) Int. Cl. A61B 17/34; A61B 17/02

(52) U.S. Cl. CPC A61B 17/3423; A61B 17/0218; A61B 2017/3466

(57) ABSTRACT

A surgical device is provided including an access port having a tubular member with a first ring secured at a proximal end and a second ring secured at a distal end; an articulation structure having an outer tube and an inner tube; and a control mechanism coupled to one end of the inner tube for advancing the inner tube through the outer tube; wherein the outer tube includes at least one rigid section and at least one flexible section and the inner tube includes at least two pre-bent sections.

### CROSS-REFERENCE TO RELATED APPLICATIONS

This application is a divisional of U.S. patent application Ser. No. 13/735,075 filed Jan. 7, 2013, which claims benefit of U.S. Provisional Application No. 61/584,713 filed Jan. 9, 2012, and the disclosures of each of the above-identified applications are hereby incorporated by reference in their entirety.

### BACKGROUND

1. Technical Field

The present disclosure relates to an apparatus and method for accessing a body cavity. More particularly, the present disclosure relates to a surgical device including an access assembly in operative association with at least one pre-bent tube.

2. Background of Related Art

Today, many surgical procedures are performed through small incisions in the skin, as compared to the larger incisions typically required in traditional procedures, in an effort to reduce both trauma to the patient and recovery time. Generally, such procedures are referred to as “endoscopic,” unless performed on the patient's abdomen, in which case the procedure is referred to as “laparoscopic.” Throughout the present disclosure, the term “minimally invasive” should be understood to encompass both endoscopic and laparoscopic procedures.

During a typical minimally invasive procedure, surgical objects, such as surgical access devices, e.g., trocar and cannula assemblies, or endoscopes, are inserted into the patient's body through the incision in tissue. In general, prior to the introduction of the surgical object into the patient's body, insufflation gasses are used to enlarge the area surrounding the target surgical site to create a larger, more accessible work area. Accordingly, the maintenance of a substantially fluid-tight seal is desirable so as to prevent the escape of the insufflation gases and the deflation or collapse of the enlarged surgical site.

To this end, various valves and seals are used during the course of minimally invasive procedures and are widely known in the art. However, a continuing need exists for a seal anchor member that can be inserted directly into the incision in tissue and that can accommodate a variety of surgical objects while maintaining the integrity of an insufflated workspace.

### SUMMARY

Accordingly, an improved surgical apparatus is provided. The surgical apparatus includes an access port having a tubular member with a first ring secured at a proximal end and a second ring secured at a distal end. The surgical apparatus further includes an articulation structure having an outer tube and an inner tube and a control mechanism coupled to one end of the inner tube for advancing the inner tube through the outer tube. The outer tube includes at least one rigid section and at least one flexible section and the inner tube includes at least two pre-bent sections.

The inner tube is configured to slidably engage and advance through the outer tube. The inner tube and the outer tube are coaxial. The inner tube defines at least one channel for receiving at least one surgical instrument.

In another exemplary embodiment, the inner tube includes at least two channels. One of the at least two channels is used for smoke evacuation from a surgical site.

In another exemplary embodiment, at least one pre-bent section of the inner tube causes a like direction bend of the flexible section of the outer tube, when the at least one pre-bent section engages the flexible section. Additionally, at least one pre-bent section of the inner tube causes the flexible section of the outer tube to bend in any direction based on rotation of the control mechanism, when the at least one pre-bent section engages the flexible section.

The control mechanism is configured to rotate the inner tube 360° degrees.

In yet another exemplary embodiment, the outer tube has two rigid sections of substantially equal length separated by the flexible section. Additionally, the outer tube has two rigid sections separated by the flexible section, at least one of which is substantially equal in length to a length of the flexible section.

In another exemplary embodiment, an improved surgical apparatus is provided. The surgical apparatus includes an access port having a tubular member with a first ring secured at a proximal end and a second ring secured at a distal end and an instrument guide device including (i) an outer member having a proximal end and a distal end, the proximal and distal ends being rigid sections connected to each other via a flexible section and (ii) an inner member having at least two rigid bends and at least one channel extending therethrough. The inner member is adapted to be inserted through and slidably engage the outer member such that at least one rigid bend of the inner member engages the flexible section of the outer member.

Also provided is an articulation method. The method includes the steps of providing an access port having a tubular member with a first ring secured at a proximal end and a second ring secured at a distal end and providing an articulation mechanism including: an outer member having a proximal end and a distal end, the proximal and distal ends being rigid sections connected to each other via a flexible section and an inner member having at least two rigid bends and at least one channel extending therethrough. The inner member is adapted to be inserted through and slidably engage the outer member such that at least one rigid bend of the inner member engages the flexible section of the outer member.

### BRIEF DESCRIPTION OF THE DRAWINGS

The accompanying drawings, which are incorporated in and constitute a part of this specification, illustrate embodiments of the disclosure and, together with a general description of the disclosure given above, and the detailed description of the embodiment(s) given below, serve to explain the principles of the disclosure, wherein:

FIG. 1 is a front perspective view of a surgical apparatus in accordance with the present disclosure shown in an expanded condition illustrating a seal anchor member positioned relative to the tissue;

FIGS. 2A-2C are side views of the outer tube shown in straight and bent configurations, in accordance with the present disclosure;

FIG. 3 is a side view of the inner tube having two pre-bent portions, in accordance with the present disclosure;

FIG. 4A is a front perspective view of the seal anchor member shown in the expanded condition and subsequent to its insertion into the incision, in accordance with the present disclosure;

FIG. 4B is a front perspective view of the seal anchor member shown in the expanded condition and subsequent to its insertion into the incision, with the inner and outer tubes inserted therethrough, in accordance with the present disclosure;

FIG. 4C is a front perspective view of the seal anchor member shown in the expanded condition and subsequent to its insertion into the incision, with the inner tube inserted entirely therethrough such that a distal end of the inner tube exits the seal anchor member, in accordance with the present disclosure;

FIG. 5A is a cross-sectional view of the tube configuration depicting an outer tube, a single inner tube slidably engaging the outer tube, and a surgical instrument inserted therethrough, in accordance with the present disclosure; and

FIG. 5B is a cross-sectional view of the tube configuration depicting an outer tube, a double slot inner tube slidably engaging the outer tube, and surgical instruments inserted therethrough, in accordance with the present disclosure.

### DETAILED DESCRIPTION

The access ports of the present disclosure, either alone or in combination with a cannula assembly, provide a substantially fluid-tight seal between a body cavity of a patient and the outside atmosphere. The access ports, or seal assemblies, of the present disclosure are configured to receive surgical instruments of varying diameter. Various surgical procedures contemplated include laparoscopic and arthroscopic surgical procedures.

The access ports of the present disclosure contemplate the introduction of various types of instrumentation adapted for insertion through a trocar and/or cannula assembly while maintaining a substantially fluid-tight interface about the instrument to help preserve the atmospheric integrity of a surgical procedure from gas and/or fluid leakage. Examples of instrumentation include, but are not limited to, clip appliers, graspers, dissectors, retractors, staplers, laser probes, photographic devices, endoscopes and laparoscopes, tubes, and the like. Such instruments will collectively be referred to as “instruments” or “instrumentation.”

Embodiments of the presently disclosed apparatus will now be described in detail with reference to the drawings, in which like reference numerals designate identical or corresponding elements in each of the several views. As used herein, the term “distal” refers to that portion of the tool, or component thereof which is further from the user while the term “proximal” refers to that portion of the tool or component thereof which is closer to the user. While the use of the access assembly is often described herein as engaging an incision, it should be recognized that this is merely exemplary and is not intended to limit the use of the assembly in any way, but rather it should be recognized that the present disclosure is intended to be useable in all instances in situations in which the access assembly engages an incision, a naturally occurring orifice, or any other suitable opening. The port is usable through an incision or through a naturally occurring orifice.

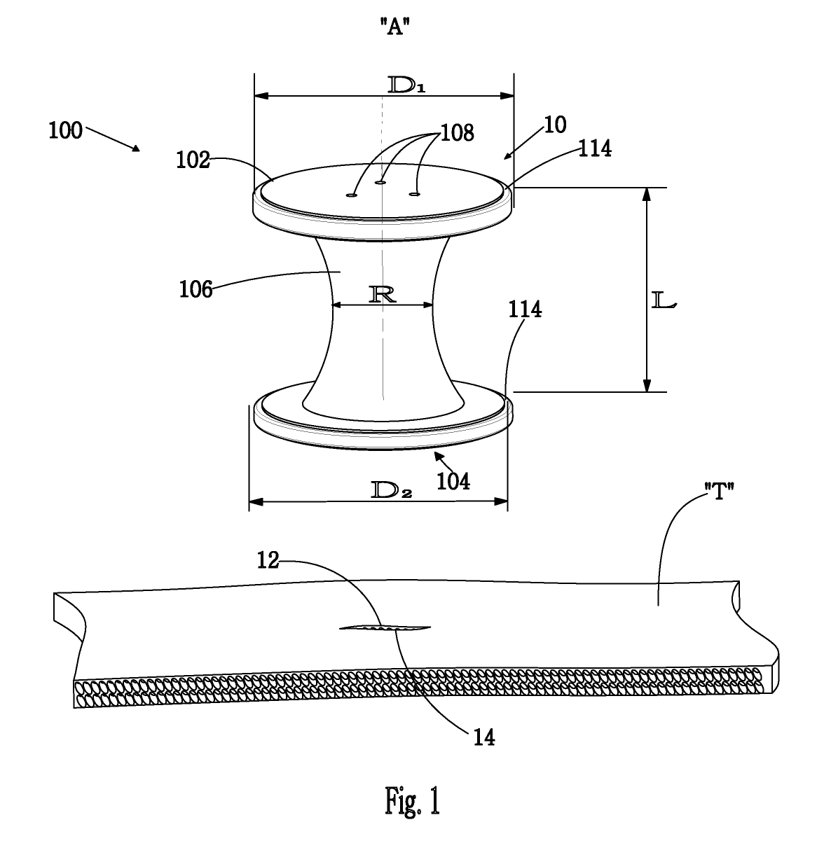

Referring to FIGS. 1-4B, a surgical apparatus 10 for use in a surgical procedure, e.g., a minimally invasive procedure is illustrated. Surgical apparatus 10 includes seal anchor member 100 (or access assembly or access port) defining a longitudinal axis “A” and having respective trailing (or proximal) and leading (or distal) ends 102, 104 and an intermediate portion 106 disposed between the trailing and leading ends 102, 104. Seal anchor member 100 includes one or more ports 108 that extend longitudinally between trailing and leading ends 102, 104, respectively, and through seal anchor member 100.

Seal anchor member 100 is preferably formed from a suitable foam material having sufficient compliance to form a seal about one or more surgical objects, and also establish a sealing relation with the tissue, “T.”

Proximal end 102 of seal anchor member 100 defines a first diameter D₁ and distal end 104 defines a second diameter D₂. In one embodiment of seal anchor member 100, the respective first and second diameters D₁, D₂ of the proximal and distal ends 102, 104 are substantially equivalent, as seen in FIG. 1, although an embodiment of seal anchor member 100 in which diameters D₁, D₂ are different is also within the scope of the present disclosure. As depicted in FIG. 1, proximal and distal ends 102, 104 define substantially planar surfaces. However, embodiments are also contemplated herein in which either or both of proximal and distal ends 102, 104, respectively, define surfaces that are substantially arcuate to assist in the insertion of seal anchor member 100 within a tissue tract 12 defined by tissue surfaces 14 and formed in tissue “T,” e.g., an incision, as discussed in further detail below.

Intermediate portion 106 defines a radial dimension “R” and extends longitudinally between proximal and distal ends 102, 104, respectively, to define an axial dimension or length “L.” The radial dimension “R” of intermediate portion 106 varies along the axial dimension, or length, “L” thereof. Accordingly, seal anchor member 100 defines a cross-sectional dimension that varies along its length “L,” which facilitates the anchoring of seal anchor member 100 within tissue “T,” as discussed in further detail below. However, an embodiment of seal anchor member 100 in which the radial dimension “R” remains substantially uniform along the axial dimension “L” thereof is also within the scope of the present disclosure.

The radial dimension “R” of intermediate portion 106 is appreciably less than the respective diameters D₁, D₂ of proximal and distal ends 102, 104 such that seal anchor member 100 defines an “hour-glass” shape or configuration to assist in anchoring seal anchor member 100 within tissue “T,” as discussed in further detail below. However, in an alternate embodiment, the radial dimension “R” of intermediate portion 106 may be substantially equivalent to the respective diameters D₁, D₂ of proximal and distal ends 102, 104. In cross section, intermediate portion 106 may exhibit any suitable configuration, e.g., substantially circular, oval or oblong.

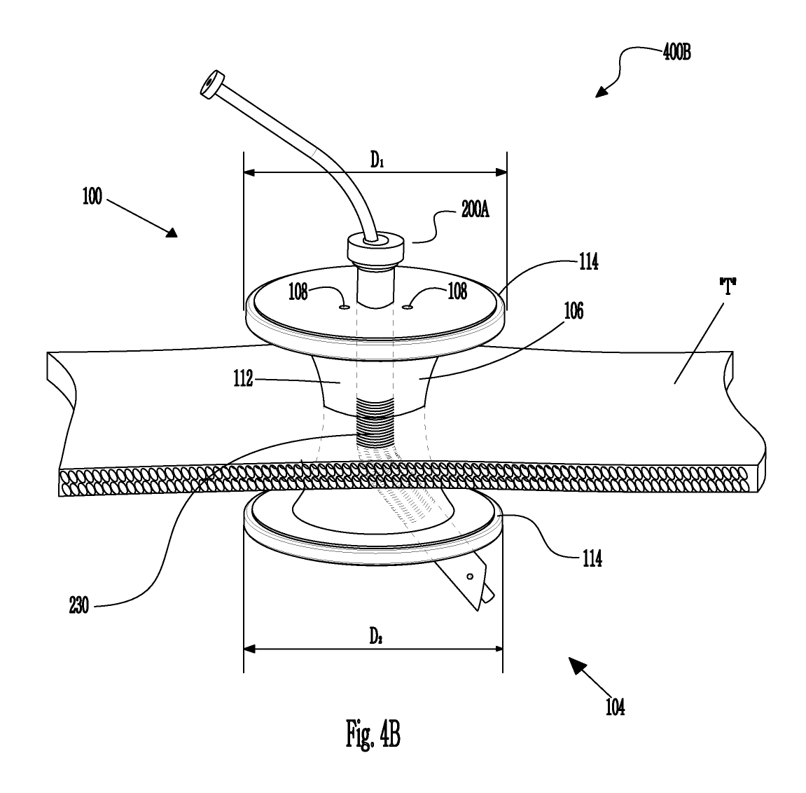

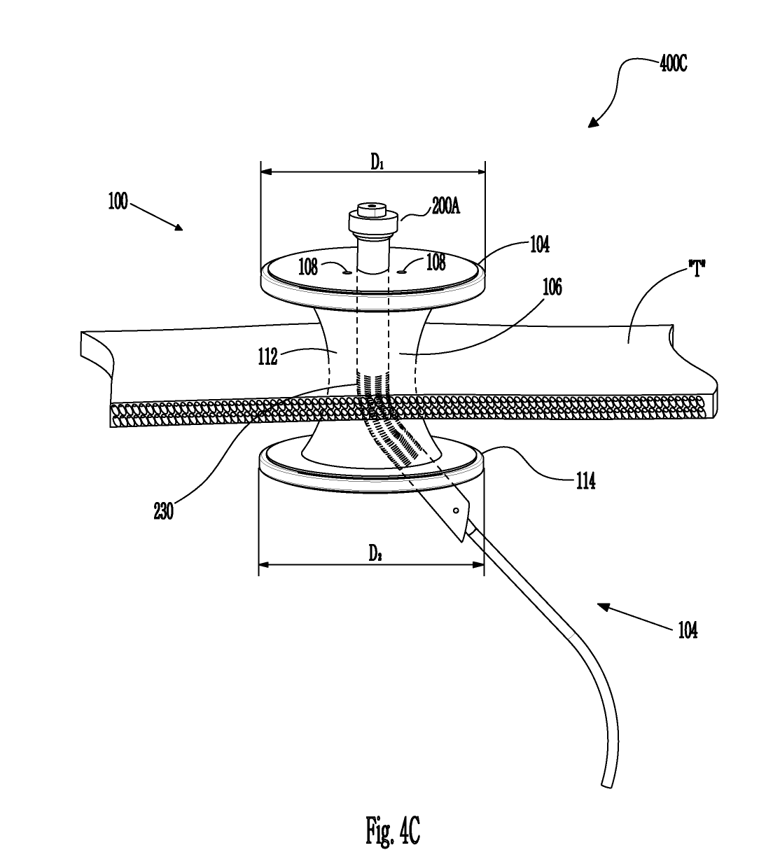

Referring now to FIGS. 1 and 4A, seal anchor member 100 is adapted to transition from an expanded condition to a compressed condition so as to facilitate the insertion and securement thereof within tissue tract 12 in tissue “T.” In the expanded condition, seal anchor member 100 is at rest and the respective radial dimensions D₁, D₂ of the proximal and distal ends 102, 104 of seal anchor member 100, as well as the radial dimension R of the intermediate portion 106 are such that the seal anchor member 100 cannot be inserted within tissue tract 12. However, in the compressed condition, proximal and distal ends 102, 104 of seal anchor member 100, as well as intermediate portion 106 are dimensioned for insertion into tissue tract 12.

Seal anchor member 100 may be formed of a biocompatible compressible material that facilitates the resilient, reciprocal transitioning of seal anchor member 100 between the expanded and compressed conditions thereof. In one embodiment, the compressible material is a “memory” foam. An external force may be applied to seal anchor member 100 to cause the seal anchor member 100 to assume the compressed condition. The external force may be directed inwardly and when seal anchor member 100 is subjected thereto, e.g., when seal anchor member 100 is squeezed, seal anchor member 100 undergoes an appreciable measure of deformation, thereby transitioning into the compressed condition.

Referring again to FIG. 1, one or more positioning members 114 may be associated with either or both of trailing (or proximal) end 102 and distal (or leading) end 104 of seal anchor member 100. Positioning members 114 may be composed of any suitable biocompatible material that is at least semi-resilient such that positioning members 114 may be resiliently deformed and may exhibit any suitable configuration, e.g., substantially annular or oval.

Prior to the insertion of seal anchor member 100, positioning members 114 are deformed in conjunction with the respective proximal and distal ends 102, 104 of seal anchor member 100 to facilitate the advancement thereof through tissue tract 12 (FIG. 4A). Subsequent to the insertion of seal anchor member 100 within tissue tract 12, the resilient nature of positioning members 114 allows positioning members to return to their normal, substantially annular configuration, thereby aiding in the expansion of either or both of the respective proximal and distal ends 102, 104 and facilitating the transition of seal anchor member 100 from its compressed condition to its expanded condition. Positioning members 114 also may engage the walls defining the body cavity to further facilitate securement of seal anchor member 100 within the body tissue. For example, positioning member 114 at leading end 104 may engage the internal peritoneal wall and positioning member 114 adjacent trailing end 102 may engage the outer epidermal tissue adjacent the incision 12 within tissue “T.” In another embodiment of seal anchor member 100, one or more additional positioning members 114 may be associated with intermediate portion 106.

In use, the peritoneal cavity (not shown) is insufflated with a suitable biocompatible gas such as, e.g., CO₂ gas, such that the cavity wall is raised and lifted away from the internal organs and tissue housed therein, providing greater access thereto. The insufflation may be performed with an insufflation needle or similar device, as is conventional in the art. Either prior or subsequent to insufflation, a tissue tract 12 is created in tissue “T,” the dimensions of which may be varied dependent upon the nature of the procedure.

Prior to the insertion of seal anchor member 100 within tissue tract 12, seal anchor member 100 is in its expanded condition in which the dimensions thereof prohibit the insertion of seal anchor member 100 into tissue tract 12. To facilitate insertion, the clinician transitions seal anchor member 100 into the compressed condition by applying a force “F” thereto, e.g., by squeezing seal anchor member 100. As best depicted in the surgical apparatus 400A of FIG. 4A, subsequent to its insertion, distal end 104, positioning member 114 and at least a section 112 of intermediate portion 106 are disposed beneath the tissue “T.” Seal anchor member 100 is caused to transition from the compressed condition to the expanded condition by removing force “F” therefrom.

After successfully anchoring seal anchor member 100 within the patient's tissue “T,” one or more surgical objects may be inserted through ports 108. FIG. 4A illustrates a surgical object introduced through one of ports 108. As previously discussed, prior to the insertion of surgical object, port 108 is in its first state in which port 108 defines an initial dimension, in one embodiment, is a longitudinal slit. Accordingly, prior to the escape of insufflation gas through port 108, in the absence of surgical object is minimal, thereby preserving the integrity of the insufflated workspace.

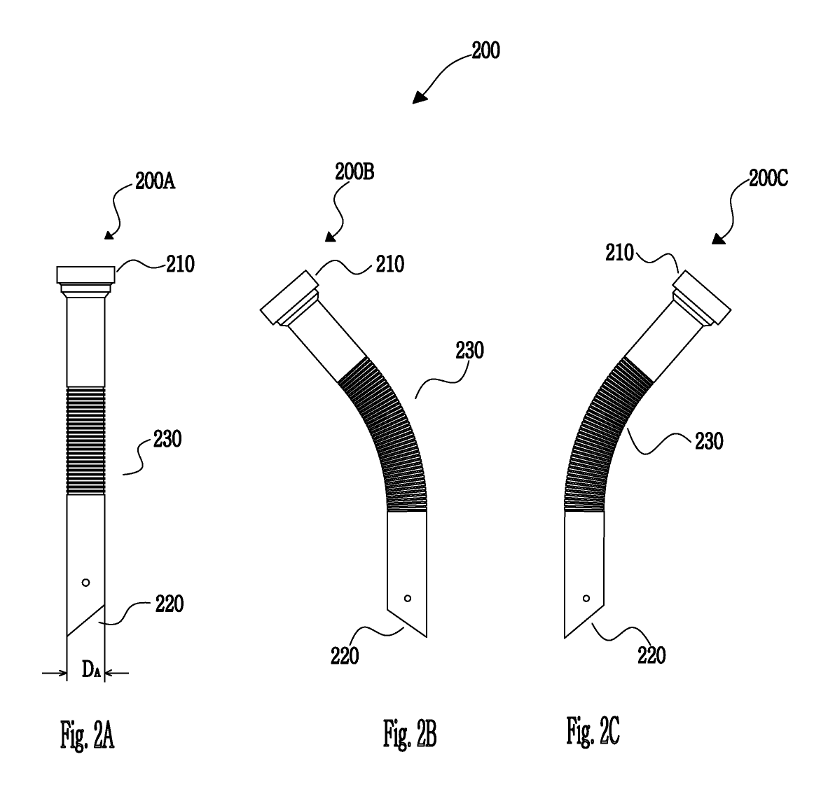

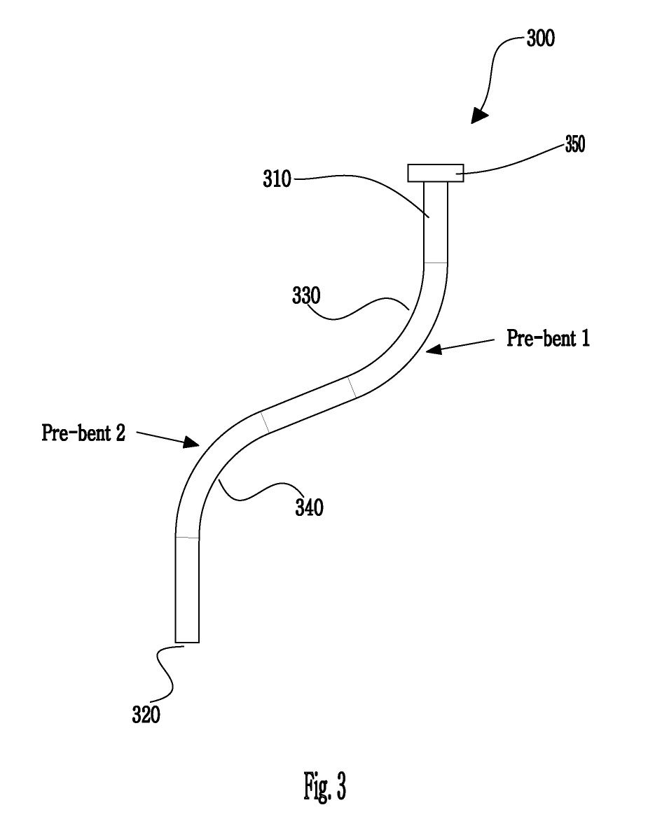

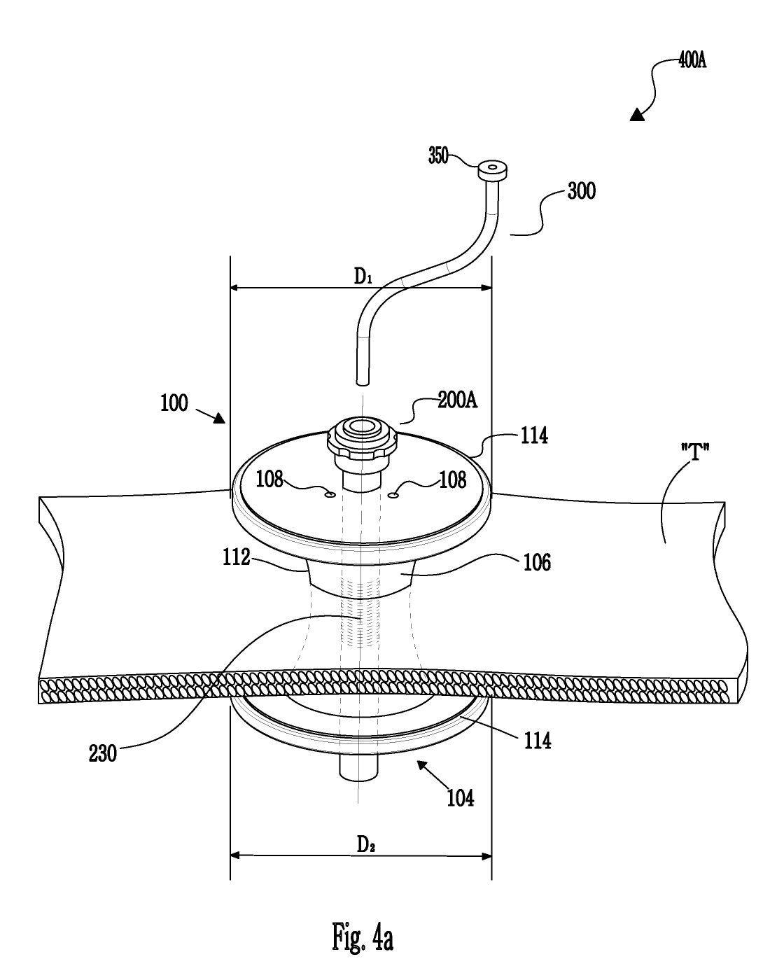

Additionally, one or more surgical objects are inserted through a tube configuration, including an outer tube and an inner tube, as described with reference to FIGS. 2A-2C and 3.

FIGS. 2A-2C illustrate an outer tube 200. FIG. 2A illustrates the outer tube 200 in a first configuration 200A being a straight configuration. FIG. 2B illustrates the outer tube 200 in a second configuration 200B being a first bent configuration. FIG. 2C illustrates the outer tube 200 in a third configuration 200C being a second bent configuration. FIGS. 2A-2C illustrate how the outer tube 200 may bend or flex either to the left (FIG. 2B) or to the right (FIG. 2C). Outer tube 200 includes a proximal end 210, a distal end 220, and a middle portion 230. The middle portion 230 is a bendable or flexible portion. Outer tube 200 may have a diameter, D_{A}. In one embodiment, it is contemplated that the proximal end 210 and the distal end 220 are rigid or semi-rigid sections or portions. It is also contemplated that the proximal portion 210, the distal portion 220, and the middle portion 230 are of substantially equal length. Therefore, the outer tube 200 may have two rigid sections 210, 220 of substantially equal length separated by the flexible section 230. However, one skilled in the art may contemplate a number of different lengths for the proximal portion 210, the distal portion 220, and the middle portion 230. For example, the middle portion 230 may be smaller that the rigid portions 210, 220.

FIG. 3 illustrates an inner tube 300 having two pre-bent portions. For example, inner tube 300 may include a proximal portion 310, a distal portion 320, a first bend 330, and a second bend 340. Thus, it is contemplated that the inner tube 300 has an “S” shaped configuration and reinforced with a rigid sleeve located between the pre-bent portions 330, 340. Additionally, the inner tube 300 may be configured to slidably engage and advance through the outer tube 200 (see FIGS. 2A-2C). As such, the inner tube 300 and the outer tube 200 may be coaxial.

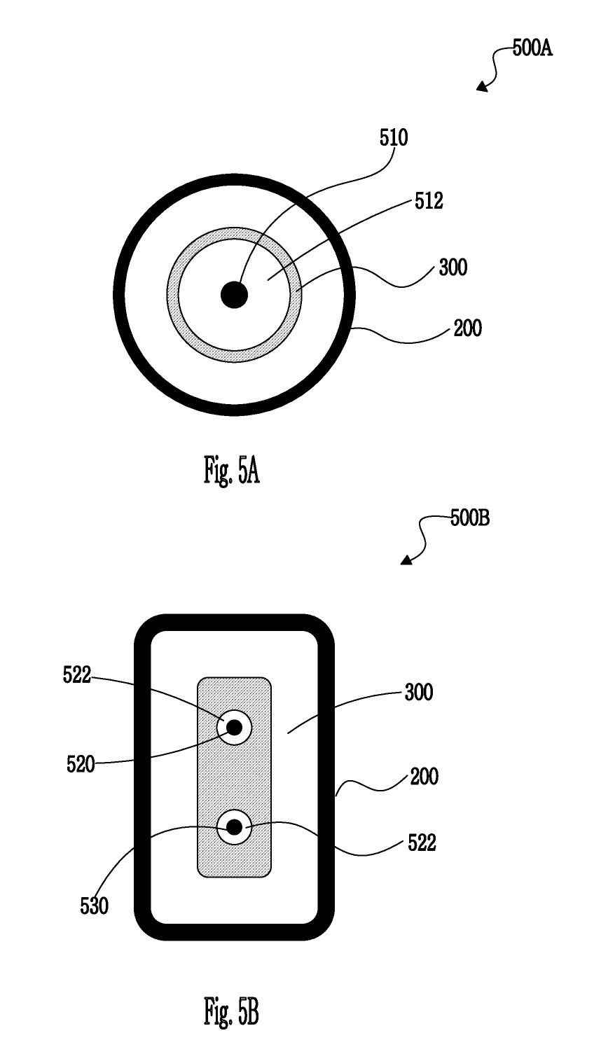

Moreover, as seen in FIGS. 5A-5B, the inner tube 300 may define at least one channel 512 for receiving at least one surgical instrument 510 as shown in FIG. 5A as a first configuration 500A. FIG. 5B illustrates an inner tube 300 having at least two channels 522, 532 for receiving two surgical instruments 520, 530, as shown in a second configuration 500B. It is contemplated that at least one of the two channels 522, 532 shown in FIG. 5B may be used for smoke evacuation from the surgical site.

Referring back to FIGS. 2A-2C and 3, at least one pre-bent section 330, 340, of the inner tube 300 may cause a like direction bend of the flexible section 230 of the outer tube 200, when the at least one pre-bent section 330, 340 engages the flexible section 230. Additionally, at least one pre-bent section 330, 340 of the inner tube 300 may cause the flexible section 230 of the outer tube 200 to bend in any direction based on rotation of a control mechanism 350, when the at least one pre-bent section 330, 340 engages the flexible section 230 of the outer tube 200. The control mechanism 350 may be configured to rotate the inner tube 300 by 360° degrees.

In operation, when the inner tube 300 is in a retracted configuration (i.e., outside the outer tube 200), an instrument is also located in a straight configuration. After insertion of the instrument into the inner tube 300, the inner tube 300 may be pushed through the outer tube 200 by a control mechanism 350. When the inner tube 300 is extended therethrough, the pre-bent section 330 moves forward and makes a left turn of articulation (see surgical apparatus 400B of FIG. 4B). At the same time, the second pre-bent section 340 moves through the flexible section 230 of the outer tube 200, thus making a right turn of articulation. In effect, the instrument is triangulated in the form of an “S” shape. In FIG. 4C, the inner tube is pushed further such that the distal portion 320 exits the distal end of the outer tube 200. The second pre-bent section 340 is shown exiting the distal end of the outer tube 200, thus changing the orientation of the flexible section 230.

When the inner tube 300 is retracted, the instrument straightens and may be removed from the outer tube 200. As such, the inner tube 300 may be easily inserted and removed to and from the outer tube 200 at any articulated positions. As a result, the motion of articulation may be determined by extending or retracting the inner tube 300 and by rotating the outer tube 200 for a 360° triangulation.

Therefore, in summary, the surgical apparatus may include an instrument guide device including (i) an outer member having a proximal end and a distal end, the proximal and distal ends being rigid sections connected to each other via a flexible section and (ii) an inner member having at least two rigid bends and at least one channel extending therethrough. The inner member is then adapted to be inserted through and slidably engage the outer member such that at least one rigid bend of the inner member engages the flexible section of the outer member. As such, at least one rigid bend of the inner member may cause a like direction bend of the flexible section of the outer member, when the at least one rigid bend engages the flexible section. Additionally, at least one rigid bend of the inner member may cause the flexible section of the outer member to bend in any direction based on rotation of a control mechanism, when the at least one rigid bend engages the flexible section. The inner member may also be connected to a control mechanism for controlling articulation of the inner member.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of presently disclosed embodiments. Thus the scope of the embodiments should be determined by the appended claims and their legal equivalents, rather than by the examples given.

Persons skilled in the art will understand that the devices and methods specifically described herein and illustrated in the accompanying drawings are non-limiting exemplary embodiments. The features illustrated or described in connection with one exemplary embodiment may be combined with the features of other embodiments. Such modifications and variations are intended to be included within the scope of the present disclosure. As well, one skilled in the art will appreciate further features and advantages of the present disclosure based on the above-described embodiments. Accordingly, the present disclosure is not to be limited by what has been particularly shown and described, except as indicated by the appended claims.

1-16. (canceled)

17. An articulation method comprising:
providing an access port having a tubular member with a first ring secured at a proximal end and a second ring secured at a distal end; and
providing an articulation mechanism including:
an outer member having a proximal end and a distal end, the proximal and distal ends being rigid sections connected to each other via a flexible section;
an inner member having at least two rigid bends and at least one channel extending therethrough;
wherein the inner member is adapted to be inserted through and slidably engage the outer member such that at least one rigid bend of the inner member engages the flexible section of the outer member.

18. The method according to claim 17, further comprising causing a like direction bend of the flexible section of the outer member via at least one rigid bend of the inner member, when the at least one rigid bend engages the flexible section.

19. The method according to claim 17, further comprising causing the flexible section of the outer member to bend in any direction based on rotation of a control mechanism via at least one rigid bend of the inner member, when the at least one rigid bend engages the flexible section.

20. The method according to claim 17, wherein the inner member includes at least two channels, one of which is used for smoke evacuation from a surgical site.

21-25. (canceled)
